(19) United States
(12) Patent Application Publication
(10) Pub. No.: US 2018/0066018 A1
(43) Pub. Date: Mar. 8, 2018

(54) CONFORMATIONALLY STABLE ANALOGS OF THE RESPONSE SELECTIVE C5A AGONIST EP67

(71) Applicant: Board Of Regents Of The University Of Nebraska, Lincoln, NE (US)

(72) Inventors: Sam D. Sanderson, Omaha, NE (US); Jonathan Vennerstrom, Omaha, NE (US)

(21) Appl. No.: 15/556,949

(22) PCT Filed: Mar. 11, 2016
(86) PCT No.: PCT/US16/22103

§ 371 Date: Sep. 8, 2017

Related U.S. Application Data
(60) Provisional application No. 62/131,393, filed on Mar. 11, 2015.

(51) Int. Cl. C07K 7/06; A61K 9/48; A61K 38/17

(52) U.S. Cl. CPC C07K 7/06; A61K 38/1745; A61K 9/48

(57) ABSTRACT

Conformationally-stable peptide analogs of the response selective C5a agonist EP67 having the formula Tyr-Ser-Phe-Lys-Asp-Met-Xaa-(Xaa2)-(D-Ala)-Arg (SEQ ID NO:1), wherein Xaa is a modified proline residue or a residue substitution for proline, and Xaa2 is leucine or N-methyl leucine. The conformationally-stable peptides selectively bind and activate APCs without directly engaging/binding C5a receptor-bearing cells involved in pro-inflammatory activities of natural C5a. Compositions and methods of using the peptide analogs are also described.

### CROSS-REFERENCE TO RELATED APPLICATIONS

The present application claims the priority benefit of U.S. Provisional Patent Application Ser. No. 62/131,393, filed Mar. 11, 2015, entitled Modulation of cis/trans Peptide Bond Isomerization in Analogs of the Response Selective C5a Agonist EP67, incorporated by reference in its entirety herein.

### SEQUENCE LISTING

The following application contains a sequence listing in computer readable format (CRF), submitted as a text file in ASCII format entitled “Sequence_Listing_47892-PCT,” created on Mar. 11, 2016, as 3 KB. The content of the CRF is hereby incorporated by reference.

### BACKGROUND OF THE INVENTION

### Field of the Invention

Conformationally-stable analogs of the response selective C-terminal analog of C5a designated as EP67.

### Description of Related Art

The blood complement (C) plays an important role in host defense to foreign substances. Individuals that are deficient in certain C components often suffer recurrent and sometimes fatal infections. Activation of the C system results in the production of the anaphylatoxins, C3a and C5a. These fragments are biologically active cleavage products of the larger C proteins C3 and C5, respectively. C5a is a short (74 residues in human) glycoprotein that is generated by enzymatic cleavage of C5.

C5a is recognized as a principal mediator of local and systemic inflammatory responses because of its ability to activate and recruit neutrophils, induce spasmogenesis, increase vascular permeability and stimulate the release of secondary inflammatory mediators from a variety of cell types (e.g., leukocytes and macrophages). C5a also plays a role in the modulation of innate and acquired immune responses because of its ability to engage and activate antigen presenting cells (APCs) to induce, directly or indirectly, the synthesis and release of the cytokines such as interleukin-1 (IL-1), interleuken-6 (IL-6), interleukin-8 (IL-8), interleuckin-12 (IL-12), and tumor necrosis factor-α (TNF-α) and enhance the antigen processing and presentation capacity of these APCs. These inflammatory and immunomodulatory activities are believed to be expressed via a transmembrane, G-protein-mediated signal transduction mechanism when the C5a ligand interacts with its receptor(s) expressed on the surface of certain circulating and tissue cell types.

The pro-inflammatory activities of C5a may be classified into two broad categories. The first category of activity (class 1) is generally associated with the release of histamines and other secondary mediators (e.g., vasoconstrictor and vasodilator eicosanoids). These activities of C5a affect many systems, and are associated with the phenomena of spasmogenesis and certain cell aggregatory activities (e.g., platelet aggregation). The second category of activity (class 2) involves recruitment and activation of neutrophils and subsequent effects of such neutrophil accumulation and activation, such as increased vascular permeability, release of cytokines and other pro-inflammatory responses. Because of its pro-inflammatory activity, C5a has been implicated as a pathogenic factor in the expression of certain inflammatory disorders, such as rheumatoid arthritis, adult respiratory distress syndrome, gingivitis, and the tissue damage associated with atherosclerosis and myocardial infarction. Consequently, considerable research efforts have been expended in developing specific C5a antagonists for use as anti-inflammatory agents in the treatment of these diseases. However, most literature relating to C5a receptor agonists and antagonists fail to differentiate between C5a receptors on C5a receptor-bearing macrophages and C5a receptors on C5a receptor-bearing granulocytes. Thus, there has been little appreciation in the art for selectively binding/activation of one type of C5a receptor over another.

U.S. Pat. Nos. 5,696,230 and 5,942,599 describe a conformational characterization of C-terminal peptide analogs of human C5a. U.S. Pat. No. 6,821,517 describes compositions and methods for delivering specific antigens to APCs via the unique C5aR that is expressed on these

APCs that differs from the C5aR expressed on inflammatory granulocytes. Co-pending U.S. 2012/0314839, filed Nov. 30, 2012 and U.S. 2015/0297668, filed Jun. 29, 2011, demonstrate advantages of selectively binding C5a receptor-bearing APCs, but not binding C5a receptor-bearing neutrophils. Each of the foregoing patents and pending applications is incorporated by reference in its entirety herein except to the extent inconsistent with the present disclosure.

### SUMMARY OF THE INVENTION

An exemplary C-terminal analog of C5a designated as EP67 (Tyr-Ser-Phe-Lys-Asp-Met-Pro-(MethylLeu)-D-Ala-Arg (YSFKDMP(MeL)aR, SEQ ID NO:2), has been demonstrated as an effective selective agonist for C5a receptor-bearing APCs. A key amino acid in this synthetic peptide analog is the proline residue at position 7, which is required for imposing an extended backbone conformation in the residue immediately to its N-terminus—at least one structural requirement that has been shown to be important for its response-selective biological activities. However, this same Pro residue undergoes rapid cis/trans isomerization and, consequently, gives rise to two populations of conformers in solution: cis/trans at ca. 25%:75%, respectively. Thus, a need exists to develop analogs of EP67, which retain its bioactivity and selectivity, but are conformationally stable.

The present invention relates to the composition and method of using a series of EP67 analogs in which this Pro is substituted with residues that impose the same/similar conformational effects of the Pro at position 7 in EP67, but lack the cis/trans isomerization associated with the Pro-7 and, consequently, results in a single population of conformers in solution. This eliminates the disruptive global structural effects that cis/trans isomerization may have on the peptide in its ability to ligate the C5aR expressed on APCs and induce the desired immunologic outcomes.

The present invention fulfills the aforementioned needs of the art by providing materials for selectively engaging/activating C5aR-bearing APCs and, consequently, exploiting their ability to induce host innate immunity for treating and preventing infectious and non-infectious diseases using an oligopeptide C-terminal analog of C5a, and also providing methods for fine tuning the conformation of oligopeptide compounds. The present invention is broadly concerned with a class of novel polypeptide products capable of eliciting favorable immune outcomes in the absence of triggering harmful inflammatory responses and the methods involved in producing these products.

We have previously described the synthesis and efficacy of a response-selective peptide agonist of C5a, EP67 in U.S. 2015/0297668. EP67 achieved many of the goals of therapy by inducing an effective immune response and sparing harmful inflammation reactions, but was prone to an unexpected cis/trans isomerization resulting in one expected compound (the trans isomer) with desirable effects, and one unexpected isomer (the cis isomer) possibly lacking these effects and/or competing with the biologically active (trans) conformer.

The result of this conversion was that a portion of the product would always be inactive or less active than that of the desired product. This instability resulted in the unavoidable presence of a product potentially capable of exhibiting unpredicted or undesired effects and leading to uncertainty about the concentration of active ingredient in dosing.

To address the stability issue of this therapeutic agent, novel peptides have been designed which exhibit all of the desirable effects of the original peptide, but eliminate the undesirable isomerization activity. These EP67 analogs are not only significantly more potent and structurally uniform than other analogs heretofore reported, but also are response-selective to elicit different classes of biological responses associated with natural C5a.

In one aspect, described herein are novel peptides that are conformationally-stable analogs of the response selective C5a agonist EP67. These peptides have the formula:

(SEQ ID NO: 1)
Tyr-Ser-Phe-Lys-Asp-Met-Xaa-(Xaa2)-(D-Ala)-Arg,

wherein Xaa is a modified proline residue or a residue substitution for proline, and Xaa2 is leucine or N-methyl leucine. The modified proline residue, when used, is one that lacks the cis/trans isomerization of unmodified proline. Advantageously, the peptide has a fixed conformation and has selective C5a receptor binding activity.

According to one aspect of the present invention, these peptides selectively elicit an immune response. In particular, the conformationally-stable peptides selectively bind and activate APCs without directly engaging/binding C5a receptor-bearing cells involved in pro-inflammatory activities of C5a (class 1 or class 2). Thus, the conformationally-stable peptides are selective agonists of C5aR-bearing APCs. In binding C5aR on APCs, the peptides activate the subject's innate immune system, which can be used to induce a non-specific immune response in the subject. The non-specific immune response can be used in the treatment of microbial infections, as well as non-infectious diseases, such as cancer and the like, discussed herein.

Likewise, in some aspects, the selectivity of the conformationally-stable peptides can be used to target a specific immunogenic agent to the APCs by functionally linking a conformationally-stable peptide to a particular immunogen (e.g., antigen). When the conformationally-stable peptide binds to the APC, the linked immunogen is internalized by the APC and generates an immune response that is specific to that immunogen. This APC-targeting utilization of peptides is described in more detail in U.S. Pat. No. 6,821,517, incorporated by reference herein.

In a further aspect, the present disclosure is also concerned with compositions comprising the inventive conformationally-stable peptides dispersed in a pharmaceutically acceptable carrier.

Also described herein are methods of inducing an immune response against infection via the selective engagement and activation of C5aR-bearing APCs in a subject. The methods generally comprise administering to the subject a therapeutically-effective amount of the inventive conformationally-stable peptides, which are response selective C5a agonists and have selective C5a receptor binding activity.

According to one aspect, the present disclosure is also concerned with kits comprising the inventive conformationally-stable peptide(s) and instructions for administering the peptide(s) to a subject in need thereof.

Uses of the inventive conformationally-stable peptide(s) are also described herein. In one aspect, the peptides are used to prepare a therapeutic or prophylactic medicament for inducing an immune response against an infection in a subject.

Peptides having the conformations and comprising the formulae set forth herein are high-potency C5a analogs that can selectively elicit different classes of biological responses associated with natural C5a. These high-potency analogs may be used as agonists to selectively elicit desired immunologic responses associated with natural C5a, and will find broad utility in treating immunocompromised patients, preferably without inflammatory side effects.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a 2-dimensional NMR analysis of EP67 in DMSO-d6 with diagnostic nuclear Overhauser effects (NOEs) evident for major (trans, ca. 80%) and minor (cis, ca. 20%) conformers; and

FIG. 2 is a series of micrographs depicting the ability of two EP67 analogs in which the Pro at position 7 has been substituted; i.e., EP144 and EP145 to drive monocyte differentiation into macrophages in vitro.

### DETAILED DESCRIPTION

The present invention is broadly concerned with a class of novel oligopeptide products capable of eliciting favorable immune outcomes through selective activation of C5a receptor-bearing APCs (e.g., macrophages, monocytes, dendritic cells), in the absence of triggering harmful inflammatory responses. In other words, the peptides selectively bind C5a receptor-bearing APCs, without binding pro-inflammatory granulocytes. The present invention relates to materials and methods for treating and preventing infectious and non-infectious disease. More specifically, the present disclosure relates to these new C5a agonist peptides, and uses thereof for treating and preventing infectious and non-infectious disease.

These agonist peptides are capable of selectively inducing innate host immune responses at the expense of inflammatory responses and thus, can be used to treat a variety of diseases including, but not limited to, microbial infections such as viral, bacterial and fungal infections; and also non-infectious diseases including, but not limited to, cancer, immune related disorders, and inflammatory disorders.

The C5a agonist peptides described in this invention can also be used to selectively induce acquired immune responses when coupled with an immunogenic agent, which can then be targeted directly to APCs through the specific binding of the peptides. In one or more embodiments, the C5a agonist peptides are covalently linked to the immunogenic agent (optionally via a spacer moiety), whereby binding of the peptide to an APC C5a receptor activates the antigen presenting cell, effecting delivery of the immunogenic agent to an antigen presenting pathway of the APC. Thus, these agonists are useful as molecular vaccine adjuvants to enhance the efficacy and immune stimulating properties of various types of vaccines. Exemplary immunogenic agents are components that resemble a disease-causing microorganism or infectious agent, and/or are made from weakened or killed forms of the same, its toxins, subunits, particles, and/or one of its surface proteins, such that it provokes an immune response in the host specific to that microorganism or infectious agent. Some vaccines contain killed, but previously virulent, microorganisms that have been destroyed. Examples include influenza, cholera, polio, hepatitis A, and rabies vaccines. Some vaccines contain live, attenuated microorganisms (modified live virus). These vaccines use live viruses that have been cultivated under conditions that disable their virulent properties, or closely related but less dangerous organisms to produce a broad immune response. Some are also bacterial in nature. Live vaccines typically provoke more durable immunological responses and in humans are the preferred type for healthy adults. Examples include measles, mumps, rubella, whooping cough, and the like. Toxoid vaccines are made from inactivated toxic compounds that cause illness rather than the microorganism itself. Examples of toxoid-based vaccines include tetanus and diphtheria. Protein subunit vaccines can also be used. In these vaccines, a fragment of the microorganism is used to create an immune response. Examples include subunit vaccines against HPV, hepatitis B, and the hemagglutinin and neuraminidase subunits of the influenza virus. Vaccines can also be formulated using viral or bacterial DNA to provoke an immune response. Furthermore, although most current vaccines are created using inactivated or attenuated compounds from microorganisms, synthetic vaccines using synthetic peptides, carbohydrates, or antigens can also be used. Cancer vaccines using tumor-specific antigens are also contemplated herein. Suitable vaccines can be monovalent or polyvalent.

The methods described in this invention can also be applied to other peptide or protein molecules to enhance or modulate the efficacy of these molecules.

The peptides are C-terminal analogs of C5a, and more specifically are conformationally stable analogs of the synthetic peptide designated as EP67 (YSFKDMP(MeL)aR (SEQ ID NO:2); where uppercase letters designate the L-stereoisomeric form and lower case the D-stereoisomeric form of the amino acids; (MeL) corresponds to N-methyl leucine). EP67 is described in detail in U.S. 2012/0314839, filed Nov. 30, 2012 and U.S. 2015/0297668, filed Jun. 29, 2011.

The present peptides are “analogs” of EP67, which, as used herein means that the peptide sequence is a variant or derivative (i.e., modified version) of the sequence of EP67 that nonetheless retains the bioactivity of EP67 (e.g., selective C5a receptor binding activity). More specifically, such “variants” or “derivatives” refer to residue substitutions or modifications made at one or two positions in the EP67 peptide sequence. Even more preferably, such substitutions or modifications occur at positions 7 and/or 8 in the EP67 sequence:

(SEQ ID NO: 2)
Tyr-Ser-Phe-Lys-Asp-Met-Pro-(MeLeu)-(D-Ala)-Arg,

where the isomerizable Pro and/or MeLeu residues are substituted or modified. These residue modifications/substitutions are counterintuitive, as the Pro and N-MeLeu residues were originally considered essential residues in the EP67 sequence. The Pro and N-MeLeu residues were originally placed in EP67 to impose an extended backbone conformation to the immediate N-terminal side of these residues, since this extended backbone conformation appeared biologically important, and more particularly aids in restricting binding of EP67 to only C5a receptor-bearing APCs and not C5a receptor-bearing granulocytes, such as inflammatory neutrophils. In one or more embodiments, the Pro residue is modified or substituted. Exemplary replacement residues include, but are not limited to, alanine, leucine, isoleucine:

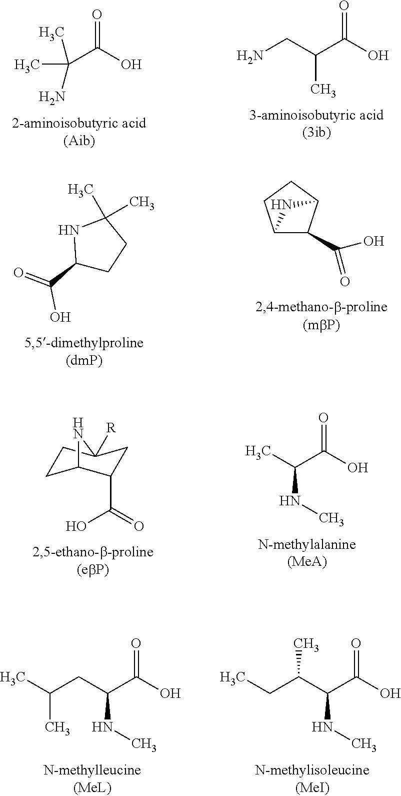

where R is —H or —CH₃.

Additional substitutions include those derived from pseudoproline, substituted pseudoproline residues, and non-N-methylated residues of the natural C5a₆₅₋₇₄ sequences of humans and other animal species. Regardless, unlike the naturally flexible C5a structure, the inventive peptide analogs are modified to be constrained in a rigid (specific) conformation, contributing to their specificity for C5aR-bearing APCs. Moreover, because cis/trans isomerization is avoided, the inventive peptides are even more constrained in terms of their 3-dimensional binding structure than EP67.

In various aspects of each embodiment of the invention, the analog is preferably 10 amino acid residues in length or less, and preferably from about 5 to about 10 amino acid residues in length. As such, in the peptide sequences described herein, the peptides are characterized by an N-terminal Tyr residue and a C-terminal Arg residue.

In one or more embodiments, the conformationally-stable EP67 analog comprises, consists essentially, or consists of Tyr-Ser-Phe-Lys-Asp-Met-Xaa-(Xaa2)-(D-Ala)-Arg (SEQ ID NO:1), wherein the original Pro residue at position 7 (Xaa) comprises singly-substituted Pro analogs at the 2, 3, 4, and/or 5 positions of the pyrrolidine side chain:

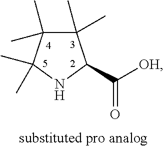

and Xaa2 is leucine or N-methyl leucine. In one or more embodiments, the EP67 analog comprises, consists essentially, or consists of doubly-substituted Pro analogs at the 2, 3, 4, and/or 5 positions of the pyrrolidine side chain and Xaa2 is leucine or N-methyl leucine. In one or more embodiments, the proline residue can be substituted with a proline homolog.

In one or more embodiments, the EP67 analog comprises, consists essentially, or consists of SEQ ID NO:1 wherein Xaa is a trifluoromethylated pseudoproline and Xaa2 is leucine or N-methyl leucine. In one or more embodiments, the EP67 analog comprises, consists essentially, or consists of SEQ ID NO:1, wherein Xaa is substituted with pseudoproline residues and Xaa2 is leucine or N-methyl leucine, where pseudoprolines Xaa(^{ΨPR1,R2}pro) are obtained by a cyclocondensation reaction of aldehydes or ketones with X=Cys, Ser, or Thr:

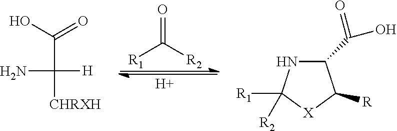

wherein:
when X═S, R═H: Cysteine-derived thiazolidines (THz);
when X═O, and R═H: Serine-derived oxazolidines (Oxa); and
when X═O, and R═CH₃: Threonine-derived oxazolidines (Oxa(5-Me)); and
R¹, R²═H, CH₃, or aryl.

In one or more embodiments, the EP67 analog comprises, consists essentially, or consists of SEQ ID NO:1, wherein Xaa is a trifluoromethylated (Tfm) azetidine 2-carboxylic acid and/or homoserine:

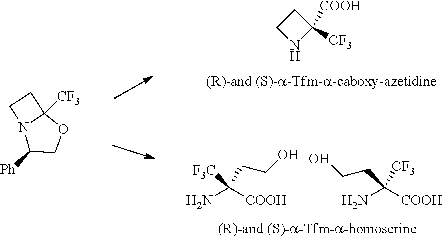 [SMILES: O=C(O)[C@@]1(C(F)(F)F)CCN1 ; FC(F)(F)C12CCN1[C@H](c1ccccc1)CO2 ; N[C@](CCO)(C(=O)O)C(F)(F)F ; N[C@@](CCO)(C(=O)O)C(F)(F)F]

and Xaa2 is leucine or N-methyl leucine.

In one or more embodiments, the EP67 analog comprises, consists essentially, or consists of SEQ ID NO:1, wherein Xaa is an oxetanyl-containing peptide:

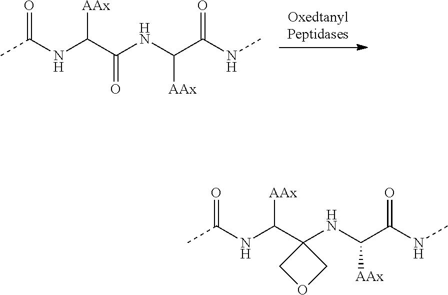

AAx stand for adjacent amino acid side chains, and Xaa is leucine or N-methyl leucine.

In one or more embodiments, the EP67 analog comprises, consists essentially, or consists of SEQ ID NO:1, wherein Xaa is an N-aminoimidazolidin-2-one (Aid) mimic of one of the following conformations (depicted as part of the larger peptide):

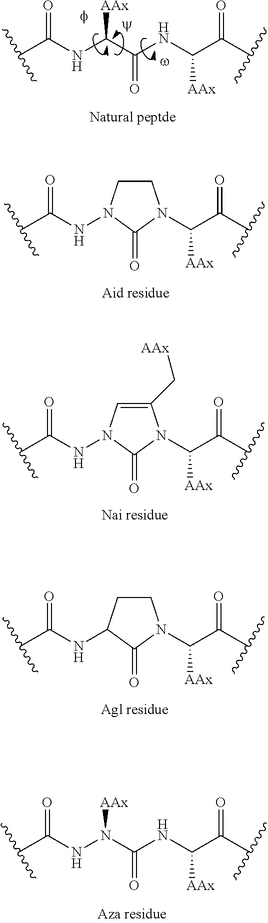

Xaa2 is leucine or N-methyl leucine, Nai is N-amino-imidazolidinone, Agl is α-amino-γ-lactam, Aza is azapeptide, and AAx stands for an amino acid side chain of the adjacent residue (Doan et al. N-Aminoimidazolidin-2-one Peptidomimetics, Org. Lett. 16, 2232-2235, 2014).

In one or more embodiments, the EP67 analog comprises, consists essentially, or consists of SEQ ID NO:1, wherein Xaa is a nonchiral pipecolic acid analog:

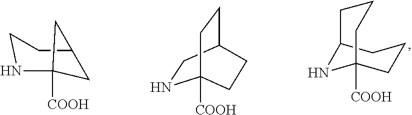 [SMILES: O=C(O)C12CC(CCN1)C2 ; O=C(O)C12CCC(CC1)CN2 ; O=C(O)C12CCCC(CCC1)N2]

and Xaa2 is leucine or N-methyl leucine.

Regardless, these synthetic proline mimetics provide restrictions of the AAx-Pro imide conformation. These proline analogs or homologs are based on ring substitutions with alkyl and aromatic groups, incorporation of heteroatoms into the ring, or the expansion or contraction of the proline ring. Exemplary proline analogs and homologs are shown in the Table below.

TABLE
Proline Analogs/Homologs

| Structure | Name |
|---|---|
| 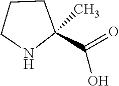 | α-methyl-L-proline |
| 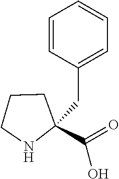 [SMILES: O=C(O)[C@]1(Cc2ccccc2)CCCN1] | α-benzyl-L-proline |
| 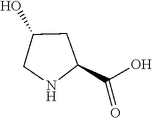 | trans-4-hydroxyl-L- proline |
| 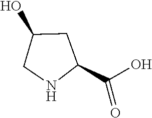 | cis-4-hydroxy-L- proline |
| 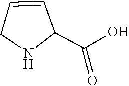 | 3,4-dehydro-DL- proline |
| 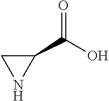 | (2S)-aziridine-2- carboxylic acid |
| 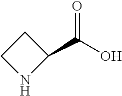 | (2S)-azetidine-2- carboxylic-acid |
| 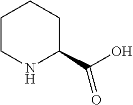 | L-pipecolic acid |
| 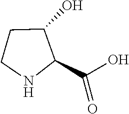 | trans-3-hydroxy-L- proline |
| 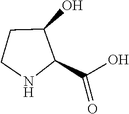 | cis-3-hydroxy-L- proline |
| 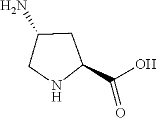 | trans-4-amino-L- proline |
| 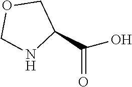 | 4-oxa-L-proline |
| 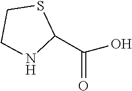 | 3-thia-DL-proline |
| 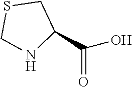 | 4-thiaL-proline |

### Source: ChemFiles, Innovations in Peptide Synthesis and Conjugation, Vol. 5, No. 12

In one or more embodiments, the EP67 analog is selected from the group consisting of

| Analog Sequence | Sequence ID Number |
|---|---|
| YSFKDM(Aib)LaR | SEQ ID NO: 3 |
| YSFKDM(Aib)(MeL)aR | SEQ ID NO: 1, where Xaa is 2- |
| | aminoisobutyric acid and Xaa2 is |
| | N-methyl leucine |
| YSFKDM(dmP)(MeL)aR | SEQ ID NO: 4 |
| YSFKDM(dmP)LaR | SEQ ID NO: 1, where Xaa is 5,5′- |
| | dimethylproline and Xaa2 is leucine |
| YSFKDM(MeL)LaR | SEQ ID NO: 1, where Xaa is N-methyl |
| | leucine and Xaa2 is leucine |
| YSFKDM(MeA)LaR | SEQ ID NO: 1, where Xaa is N- |
| | methylalanine and Xaa2 is leucine |
| YSFKDMQ(MeL)aR | SEQ ID NO: 1, where Xaa is glycine |
| | and Xaa2 is N-methyl leucine |
| YSFKDM(Pip)(MeL)aR | SEQ ID NO: 1, where Xaa is pipecolic |
| | analog and Xaa2 is N-methyl leucine |
| YSFKDM(Pip)LaR | SEQ ID NO: 1, where Xaa is pipecolic |
| | analog and Xaa2 is leucine |
| YSFKDM(ΨP)(MeL)aR | SEQ ID NO: 1, where Xaa is pseudoproline |
| | and Xaa2 is N-methyl leucine |
| YSFKDM(ΨP)LaR | SEQ ID NO: 1, where Xaa is pseudoproline |
| | and Xaa2 is leucine |
| YSFKDM(eβP)(MeL)aR | SEQ ID NO: 1, where Xaa is 2,5-ethano-β- |
| | proline and Xaa2 is N-methyl leucine |
| YSFKDM(MeL)(MeL)aR | SEQ ID NO: 1, where Xaa is N-methyl |
| | leucine and Xaa2 is N-methyl leucine |
| YSFKDM(3ib)(MeL)aR | SEQ ID NO: 1, where Xaa is 3- |
| | aminoisobutyric acid and Xaa2 is |
| | N-methyl leucine |
| YSFKDM(mβP)(MeL)aR | SEQ ID NO: 1, where Xaa is 2,4-methano-β- |
| | proline and Xaa2 is N-methyl leucine |
| YSFKDM(MeA)(MeL)aR | SEQ ID NO: 1, where Xaa is N- |
| | methylalanine and Xaa2 is N- |
| | methyl leucine |
| YSFKDM(MeI)(MeL)aR | SEQ ID NO: 1, where Xaa is N- |
| | methylisoleucine and Xaa2 is N- |
| | methyl leucine |

Aib=2-aminoisobutyric acid; 3ib=3-aminoisobutyric acid; dmP=5,5′-dimethylproline; mβP=2,4-methano-β-proline; eβP=2,5-ethano-β-proline; MeA=N-methylalanine; MeL=N-methylleucine; MeI=N-methylisoleucine; Pip=pipecolic acid derivatives/analogs including but not limited to:

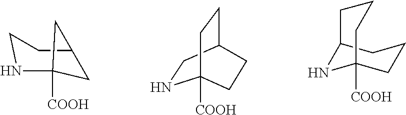 [SMILES: O=C(O)C12CC(CCN1)C2 ; O=C(O)C12CCC(CC1)CN2 ; O=C(O)C12CCCC(CCC1)N2]

ΨP=serine/threonine/cysteine-derived pseudoproline analogs including but not limited to

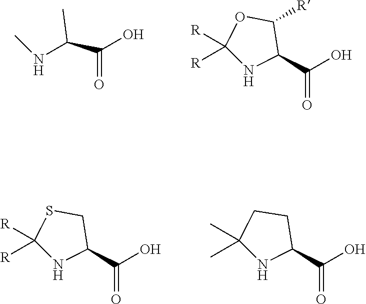

where R═H or CH₃; R′═CH₃ (Theonine-derived) or R′═H (Serine-derived).

Particularly preferred conformationally-stable EP67 analogs include YSFKDM(Aib)LaR (SEQ ID NO:3) and YSFKDM(dmP)(MeL)aR (SEQ ID NO:4)

The conformationally-stable EP67 analogs are used to induce innate and acquired immune responses while sparing inflammation. Advantageously, the present invention allows for the use of a lower therapeutic dose with increased C5aR binding affinity on APCs, and bioselectivity, thereby preventing side effects resulting from the non-binding analog conformer.

Compositions comprising the conformationally-stable peptide analogs are also described herein. In various embodiments, the composition comprises a pharmaceutically acceptable carrier. The term carrier is used herein to refer to diluents, excipients, vehicles, coatings and the like, in which the peptide(s) may be dispersed or coated with for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would naturally be selected to minimize any degradation of the compound or other agents and to minimize any adverse side effects in the subject, as would be well known to one of skill in the art. Pharmaceutically-acceptable ingredients include those acceptable for veterinary use as well as human pharmaceutical use, and will depend on the route of administration. Any carrier compatible with the excipient(s) and EP67 analogs can be used. Supplementary active compounds may also be incorporated into the compositions.

A composition of the present disclosure is formulated to be compatible with its intended route of administration. Examples of routes of administration include oral administration (ingestion) and parenteral administration, e.g., intravenous, intradermal, subcutaneous, inhalation, nasal, transdermal (topical), transmucosal, buccal, sublingual, pulmonary and rectal administration.

Pharmaceutical compositions suitable for injectable use include sterile aqueous solutions (where water-soluble), solutions in sterile isotonic aqueous buffer, or dispersions and sterile powders for the extemporaneous preparation of sterile injectable solutions or dispersion. For intravenous administration, suitable carriers include physiological saline, Cremophor EL™ (BASF, Parsippany, N.J.), bacteriostatic/sterile water/distilled autoclaved water (DAW), or phosphate buffered saline (PBS). In all cases, the composition is sterile and fluid to allow syringability. The carrier may be a solvent or dispersion medium containing, for example, water, ethanol, polyol (for example, glycerol, propylene glycol, and liquid polyethylene glycol, and the like), and suitable mixtures thereof. Fluidity is maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions may be brought about by including in the composition an agent that delays absorption, for example, aluminum monostearate and gelatin. The injectable preparations may be enclosed in ampules, disposable syringes or multiple dose vials made of glass or plastic.

Solutions or suspensions used for parenteral application (injection or infusion) may include the following components: a sterile diluent such as water for injection, saline solution, fixed oils, polyethylene glycols, glycerin, propylene glycol, various oil-in-water or water-in-oil emulsions, as well as dimethyl sulfoxide (DMSO), or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH may be adjusted with acids or bases, such as hydrochloric acid or sodium hydroxide.

Oral compositions generally include an inert diluent or an edible carrier. Oral formulations generally take the form of a pill, tablet, capsule (e.g., soft gel capsule, solid-filled capsule, or liquid-filled capsule), solid lozenge, liquid-filled lozenge, mouth and/or throat drops or spray, effervescent tablets, orally disintegrating tablet, suspension, emulsion, syrup, elixir, or tincture. The composition may be contained in enteric forms to survive the stomach or further coated or mixed to be released in a particular region of the gastrointestinal tract by known methods. Solid oral dosage forms are typically swallowed immediately, or slowly dissolved in the mouth. Oral compositions may also be prepared using a fluid carrier for use as a mouthwash, wherein the compound in the fluid carrier is applied orally and swished and expectorated or swallowed. Oral formulations optionally contain any of the following ingredients, or compounds of a similar nature: a binder such as microcrystalline cellulose, gum tragacanth or gelatin; starch or lactose; a disintegrating agent such as alginic acid, Primogel™, or corn starch; a lubricant such as magnesium stearate; a glidant such as colloidal silicon dioxide; and/or a sweetening agent such as sucrose or saccharin.

For administration by inhalation, the composition is optionally delivered in the form of a spray. The spray may be an aerosol spray from a pressured container or dispenser, which contains a suitable propellant, e.g., a gas such as carbon dioxide, or a nebulizer. The composition is optionally formulated for delivery via a dry powder inhaler (DPI), a metered dose inhaler (pMDI), nasal spray, or a vaporizer. For routes of administration involving absorption of an agent and/or excipient through mucosal membrane, the composition further optionally comprises a penetrant.

Optionally, the composition is formulated as a “liquid respiratory composition,” i.e., a composition in a form that is deliverable to a mammal via the oral cavity, mouth, throat, nasal passage or combinations thereof. These compositions can be delivered by a delivery device selected from droppers, pump, sprayers, liquid dropper, spoon, cup, squeezable sachets, power shots, and other packaging and equipment, and combinations thereof. In one embodiment, the liquid respiratory composition comprises the therapeutic agent, and excipient, a thickening polymer (e.g., xanthan gum, cellulosic polymers such as carboxymethycellulose (CMC), hydroxethylcellulose, hydroxymethylcellulose, and hydroxypropylmethylcellulose, carrageenan, polyacrylic acid, cross-linked polyacrylic acid such as Carbopol®, polycarbophil, alginate, clay, and combinations thereof), and optionally a mucoadhesive polymer (e.g., polyvinylpyrrolidone (Povidone), methyl vinyl ether copolymer of maleic anhydride (Gantrez®), guar gum, gum tragacanth, polydextrose, cationic polymers, poly(ethylene oxide), poly(ethylene glycol), poly(vinyl alcohol), poly(acrylic acid), cross-linked polyacrylic acid such as Carbopol®, polycarbophil, poly(hydroxyl ethyl methacrylate), chitosan, cellulosic polymers such as carboxymethycellulose (CMC), hydroxethylcellulose, hydroxymethylcellulose, and hydroxypropylmethylcellulose, and combinations thereof). The composition is preferably a non-Newtonian liquid that exhibits zero shear viscosity from about 100 centiPoise (cP) to about 1,000,000 cP, from about 100 cP to about 500,000 cP, from about 100 cP to about 100,000 cP, from about 100 cP to about 50,000 cP, from about 200 cP to about 20,000 cP, from about 1,000 to about 10,000 cP at a temperature of about 37° C., as measured according to the Shear Viscosity Method. The pH range of the formulation is generally from about 1 to about 7, from about 2 to about 6.5, and from about 4 to about 6.

In general additional pharmaceutically-acceptable ingredients for use in the compositions include adjuvants, antigens, buffering agents, salts, stabilizing agents, diluents, preservatives, antibiotics, isotonic agents, cell media (e.g., MEM, FBS), flavoring agents, and the like. Exemplary isotonic agents include dextrose, lactose, sugar alcohols (e.g., sorbitol, mannitol), and the like. Stabilizing agents include sugars such as sucrose and lactose, amino acids such as glycine or the monosodium salt of glutamic acid and proteins such as albumin or gelatin, and mixtures thereof. Exemplary preservatives include formaldehyde, thimerosal, and the like.

In various embodiments, in addition to the carrier and peptide analogs described herein, a nasal spray formulation may comprise benzalkonium chloride, camphor, chlorhexidine gluconate, citric acid, disodium EDTA, eucalyptol, menthol, purified water, and/or tyloxapol. An exemplary oral composition may comprise FD&C Blue No. 1, gelatin, glycerin, polyethylene glycol, povidone, propylene glycol, purified water, sorbitol special, and/or titanium dioxide in addition to an excipient and acetaminophen, doxylamine succinate, and phenylephrine HCl (or dextromethorphan).

In various embodiments, powders, creams and gels are contemplated for topical administration of a pharmaceutical composition. In one embodiment, the topical administration refers to the application of a therapeutic composition to a localized area of the body or to the surface of a body part (e.g., on the skin) where action or symptom relief is desired. In one embodiment, a transdermal patch is used according the present disclosure. In still other embodiments, a pharmaceutical composition according to the present disclosure is embedded, e.g., in wound dressings, bandages (e.g., hydrocolloids, hydrogels, alginates, foams, gauze), and/or surgical sutures to prevent and/or treat infections and improve wound (e.g., scrapes, cuts, and surgical incisions) healing.

In one embodiment, the components of the composition are prepared with carriers that will protect the components against rapid elimination from the body, such as a controlled release formulation, including coatings, implants, and microencapsulated delivery systems. Biodegradable, biocompatible polymers may be used, such as ethylene vinyl acetate, polyanhydrides, polyglycolic acid, collagen, polyorthoesters, and polylactic acid.

The formulation is provided, in various aspects, in unit dosage form for ease of administration and uniformity of dosage. “Unit dosage form” as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms are dictated by and are directly dependent on the unique characteristics of the excipient(s) and therapeutic agent(s) and the particular biological effect to be achieved.

Safety and efficacy of compositions described herein are determined by standard procedures using in vitro or in vivo technologies, such as the materials and methods described herein and/or known in the art. Administration may be on an as-needed or as-desired basis, for example, once-monthly, once-weekly, or daily, including multiple times daily, for example, at least once daily, from one to about ten times daily, from about two to about four times daily, or about three times daily. A dose of composition optionally comprises about from about 0.001 mg to about 1000 mg active agent, alternatively from about 2.5 mg to about 750 mg active agent, and alternatively from about 5 mg to about 650 mg of the active agent. In one embodiment, a dose of composition according to the present disclosure comprises about from 0.1 mg to about 0.25 mg. In various embodiments, a dose of composition according to the present disclosure comprises 25 μg, 50 μg, 60 μg, 70 μg, 80 μg, 90 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg or 500 μg. In various embodiments, a dose of composition according to the present disclosure comprises between 25 μg to 500 μg, 50 μg to 400 μg, 100 μg to 300 μg, or 200 μg to 250 μg.

In various embodiments, the conformationally-stable EP67 analogs or a pharmaceutical composition comprising the conformationally-stable EP67 analogs, is used in combination with one or more other active agents useful for treating or preventing infections or diseases. The other active agent(s) can enhance the effects of the therapeutic agent and/or exert other pharmacological effects in addition to those of the therapeutic agent. Non-limiting examples of active agents that can be used in combination with a therapeutic agent are immunosuppressants (e.g., cyclosporine, azathioprine), corticosteroids, anti-inflammatory agents, chemotherapeutic agents, antibiotics, antifungals, antivirals and antiparasitics. As described herein, other exemplary active agents that are contemplated include vaccines (e.g., existing vaccines directed to a specific pathogen or disease) and vaccines comprising C-terminal analogs of C5a conjugated to a specific antigen.

The compositions described herein can be used as part of a treatment for a variety of diseases including, but not limited to, microbial infections such as viral, bacterial and fungal infections; these compositions can also be used to treat non-infectious diseases including, but not limited to, cancer, immune related disorders, and inflammatory disorders. The compositions described in this invention can also be used as vaccine adjuvants to enhance the efficacy and immune stimulating properties of various types of vaccines.

In use, a therapeutically-effective amount of a conformationally-stable EP67 analog is administered to a subject. Administration of the conformationally-stable EP67 analog elicits an immune response in the subject, and more specifically a selective activation of the innate immune response, without direct activation of pro-inflammatory neutrophils and other granulocytes. The immune response will be demonstrated by a lack of observable clinical symptoms, or reduction of clinical symptoms normally displayed by an infected subject, faster recovery times from infection, reduced duration of infection, and the like. In another embodiment, a method of activating an immune cell at a site of infection or disease is provided comprising administering an effective amount of the conformationally-stable EP67 analog to a mammal, said analog having selective C5a receptor binding activity. It will be appreciated that although the conformationally-stable EP67 analog does not directly bind or activate the pro-inflammatory granulocytes, a secondary inflammatory response may be initiated due to the release of chemokines/cytokines by the APCs once activated by the peptide analogs.

In various aspects of each embodiment of the disclosure, the infection or disease is caused by an infectious agent selected from the group consisting of bacteria, virus, fungus, parasite, protozoan, and prion. In other various aspects of each embodiment, the disease is cancer. In various aspects of each embodiment of the method, the infection comprises a biofilm.

In various aspects of each embodiment of the disclosure involving a bacterial infection, the bacteria is selected from the group consisting of methicillin-resistant S. aureus (MRSA), MRSA strain USA300-FPR3757, vancomycin-resistant S. aureus (VRSA), macrolide-resistant S. pyogenes, penicillin-resistant Streptococcus pneumoniae, Extensively Drug-Resistant Mycobacterium tuberculosis (XDR TB), multidrug-resistant Enterococcus faecalis, multidrug-resistant Enterococcus faecium, Pseudomonas aeruginosa, clindamycin-resistant Clostridium difficile, fluoroquinolone-resistant Clostridium difficile, Acinetobacter baumannii, Bacillus anthracis, Bordetella pertussis, Borrelia burgdorferi, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostridium tetani, Corynebacterium diphtherias, Enterococcus faecalis, Enterococcus faecium, Escherichia coli, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

In various aspects of each embodiment of the disclosure involving a viral infection, the virus is selected from the group consisting of Poxviridae, Chordopoxvirinae, Orthopoxvirus, Cowpoxvirus, Monkeypox virus, Vaccinia virus, Variola virus, Parapoxvirus, Bovine papular stomatitis virus, Orf virus, Pseudocowpox virus, Molluscipoxvirus, Molluscum contagiosum virus, Yatapoxvirus, Tanapox virus, Yaba monkey tumor virus, Herpesviridae, Alphaherpesvirinae, Simplexvirus, Human herpesvirus 1, Herpes simplex virus 1, Human herpesvirus 2, Herpes simplex virus 2, Varicellovirus, Human herpesvirus 3, Varicella-zoster virus, Betaherpesvirinae, Cytomegalovirus, Human herpesvirus 5, Human cytomegalovirus, Roseolovirus, Human herpesvirus 6, Human herpesvirus 7, Gammaherpesvirinae, Lymphocryptovirus, Human herpesvirus 4, Epstein-Barr virus, Rhadinovirus, Human herpesvirus 8, Kaposi's sarcoma-associated herpesvirus, Adenoviridae, Mastadenovirus, Human adenovirus A, Human adenovirus B, Human adenovirus C, Human adenovirus D, Human adenovirus E, Human adenovirus F, Polyomaomaviridae, Polyomavirus, BK polyomavirus , Human polyomavirus, JC polyomavirus, Papillomaviridae, Alphapapillomavirus, Human papillomavirus 2, Human papillomavirus 10, Human papillomavirus 6, Human papillomavirus 7, Human papillomavirus 16, Human papillomavirus 18, Human papillomavirus 26, Human papillomavirus 32, Human papillomavirus 34, Human papillomavirus 53, Human papillomavirus 54, Human papillomavirus 61, Human papillomavirus 71, Human papillomavirus cand90, Betapapillomavirus, Human papillomavirus 5, Human papillomavirus 9, Human papillomavirus 49, Human papillomavirus cand92, Human papillomavirus cand96, Gammapapillomavirus, Human papillomavirus 4, Human papillomavirus 48, Human papillomavirus 50, Human papillomavirus 60, Human papillomavirus 88, Mupapillomavirus, Human papillomavirus 1, Human papillomavirus 63, Parvoviridae, Parvovirinae, Erythrovirus, B19 virus, Hepadnaviridae, Orthohepadnavirus, Hepatitis B virus, Retroviridae, Orthoretrovirinae, Deltaretrovirus, Primate T-lymphotropic virus 1, Primate T-lymphotropic virus 2, Lentivirus, Human immunodeficiency virus 1, Human immunodeficiency virus 2, Reoviridae, Orthoreovirus, Mammalian orthoreovirus, Orbivirus, African horse sickness virus, Changuinola virus, Corriparta virus, Orungo virus, Rotavirus, Rotavirus A, Rotavirus B, Mononegavirales, Filoviridae, Marburgvirus, Lake Victoria marburgvirus, Ebolvirus, Ivory Coast ebolavirus, Reston ebolavirus, Sudan ebolavirus, Zaire ebolavirus, Paramyxoviridae, Paramyxovirinae, Respirovirus, Human parainfluenza virus 1, Human parainfluenza virus 3, Morbillivirus, Measles virus, Edmonston virus, Rubulavirus, Human parainfluenza virus 2, Human parainfluenza virus 4, Mumps virus, Henipavirus, Hendravirus, Nipahvirus, Pneumovirinae, Pneumovirus, Human respiratory syncytial virus, Metapneumovirus, Human metapneumovirus, Rhabdoviridae, Vesiculovirus, Chandipura virus, Cocal virus, Isfahan virus, Piry virus, Vesicular stomatitis Alagoas virus, Vesicular stomatitis Indiana virus, Vesicular stomatitis New Jersey virus, Lyssavirus, Australian bat lyssavirus, Rabies virus, Orthomyxoviridae, Influenzavirus A, Influenza A virus, Influenzavirus B, Influenza B virus, Influenzavirus C, Influenza C virus, Bunyaviridae, Bunyavirus, Bunyamwera virus, Bwamba virus, California encephalitis virus, Guama virus, Oriboca virus, Oropouche virus, Hantavirus, Andes virus, Hantaan virus, Puumala virus, Seoul virus, Dobrava-Belgrade virus, Bayou virus, Black Creek Canal virus, New York virus, Sin Nombre virus, Nairovirus, Crimean-Congo hemorrhagic fever virus, Nairobi sheep disease virus, Phlebovirus, Rift Valley fever virus, Sandfly fever Naples virus, Arenaviridae, Arenavirus, Lassa virus, Lymphocytic choriomeningitis virus, Guanarito virus, Junin virus, Machupo virus, Sabia virus, Deltavirus, Hepatitis delta virus, Nidovirales, Coronaviridae, Coronavirus, Human coronavirus 229E, Human coronavirus OC43, Human enteric coronavirus, Severe acute respiratory syndrom coronavirus, Torovirus, Picornaviridae, Enterovirus, Human enterovirus A, Human enterovirus B, Human enterovirus C, Human enterovirus D, Poliovirus, Rhinovirus, Human rhinovirus A, Human rhinovirus B, Hepatovirus, Hepatitis A virus, Parechovirus, Human parechovirus, Caliciviridae, Norovirus, Norwalk virus, Sapovirus, Sapporo virus, Hepevirus, Hepatitis E virus, Astroviridae, Mamastrovirus, Human astrovirus, Togaviridae, Alphavirus, Chikungunya virus, O'nyong-nyong virus, Mayaro virus, Ross River virus, Barmah Forest virus, Sindbis virus, Ockelbo virus, Venezuelan equine encephalitis virus, Western equine encephalitis virus, Eastern equine encephalitis virus, Rubivirus, Rubella virus, Flaviviridae, Flavivirus, Kyasanur Forest disease virus, Omsk hemorrhagic fever virus, Powassan virus, Louping ill virus, Tick-borne encephalitis virus, Dengue virus, Japanese encephalitis virus, Murray Valley encephalitis virus, St. Louis encephalitis virus, West Nile virus, Ilheus virus, Yellow fever virus, Apoi virus, Hepacivirus, Hepatitis C virus, GB virus B, and GB virus A.

In various aspects of each embodiment involving a fungus, the fungus is selected from the group consisting of C. albicans, A. fumigates, A. flavus, A. clavatus, C. neoformans, C. laurentii, C. albidus, C. gatti, H. capsulatum, P. jirovecii, S. chartarum, C. immitis and C. posadasii.

In various aspects of each embodiment involving a parasite, the parasite is selected from the group consisting of protozoans, helminthes, parasitic worms, Halzoun syndrome, myiasis, Chogoe fly, human botfly, candiru, bedbug, head louse, body louse, crab louse, demodex, scabies, and screwworm.

In various aspects of each embodiment involving a protozoan, the protozoan is selected from the group consisting of Entamoeba Histolytica, Giardia Lambila, Trichomonas Vaginalis, Trypanosoma Brucei, T Cruzi, Leishmania Donovani, Balantidium Coli, Toxoplasma Gondii, Plasmodium Spp., and Babesia Microti.

In various aspects of each embodiment of the disclosure, the disease is selected from the group consisting of scrapie, bovine spongiform encephalopathy, transmissible mink encephalopathy, chronic wasting disease, feline spongiform encephalopathy, exotic ungulate encephalopathy, Creutzfeldt-Jakob disease, iatrogenic Creutzfeldt-Jakob disease, variant Creutzfeldt-Jakob disease, familial Creutzfeldt-Jakob disease, sporadic Creutzfeldt-Jakob disease, Gerstmann-Straussler-Scheinker syndrome, fatal familial insomnia, and Kuru.

To achieve a desired therapeutic outcome in a combination therapy, a conformationally-stable EP67 analog and other active agent(s) are generally administered to a subject in a combined amount effective to produce the desired therapeutic outcome (e.g., reduction or elimination of one or more symptoms). The combination therapy can involve administering the conformationally-stable EP67 analogs and the other active agent(s) at about the same time. Simultaneous administration can be achieved by administering a single composition that contains both the conformationally-stable EP67 analogs and the other active agent(s). Alternatively, the other active agent(s) can be taken separately at about the same time as a pharmaceutical formulation comprising the conformationally-stable EP67 analogs (i.e., sequentially). In either case, the active agent and EP67 analog are considered to have been “co-administered.”

In other alternatives, administration of the conformationally-stable EP67 analogs can precede or follow administration of the other active agent(s) by an interval ranging from minutes to hours. In embodiments where the conformationally-stable EP67 analogs and the other active agent(s) are administered at different times, the conformationally-stable EP67 analogs and the other active agent(s) are administered within an appropriate time of one another so that both the conformationally-stable EP67 analogs and the other active agent(s) can exert a beneficial effect (e.g., synergistically or additively) on the recipient. In some embodiments, the conformationally-stable EP67 analogs is administered to the subject within about 0.5-12 hours (before or after), or within about 0.5-6 hours (before or after), of the other active agent(s). In certain embodiments, the conformationally-stable EP67 analogs is administered to the subject within about 0.5 hour or 1 hour (before or after) of the other active agent(s).

A “booster” dose of a conformationally-stable EP67 analogs or a pharmaceutical composition comprising a conformationally-stable EP67 analogs, separately or in combination with another active agent as described above, is also contemplated by the present disclosure. A booster dose may be administered about 1 week, about 1 month, about 2 months, about 3 months, about 4 months, about 5 months, about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years, about 15 years, and about 20 years after an initial administration.

A kit comprising the conformationally-stable EP67 analog is also disclosed herein. The kit further comprises instructions for administering the conformationally-stable EP67 analog to a subject. The conformationally-stable EP67 analog(s) can be provided as part of a dosage unit, already dispersed in a pharmaceutically-acceptable carrier, or it can be provided separately from the carrier. The kit can further comprise instructions for preparing the conformationally-stable EP67 analog for administration to a subject, including for example, instructions for dispersing the analog(s) in a suitable carrier.

It will be appreciated that therapeutic and prophylactic methods described herein are applicable to humans as well as any suitable animal, including, without limitation, dogs, cats, and other pets, as well as, rodents, primates, horses, cattle, pigs, etc. The methods can be also applied for clinical research and/or study.

As described herein, the ability to induce innate immunity in a non-antigen-specific method has advantages in that it affords induction of immune responses to a wide range of pathogens irrespective of the nature of the antigens these pathogens express. Thus, the ability to induce a protective immune response is not dependent upon reaction to a specific antigen expressed by a pathogen, but rather to the pathogen itself.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

### General Definitions

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Thus, for example, the reference to a particular C-terminal analog of C5a is a reference to one such analog or a plurality of such analogs, including equivalents thereof. Also, the terms “at least one” and “one or more” have the same meaning and include one, two, three or more. The following terms, unless otherwise indicated, shall be understood to have the following meanings when used in the context of the present disclosure.

Examples provided herein, including those following “such as” and “e.g.,” are considered as illustrative only of various aspects of the present disclosure and embodiments thereof, without being specifically limited thereto. Any suitable equivalents, alternatives, and modifications thereof (including materials, substances, constructions, compositions, formulations, means, methods, conditions, etc.) known and/or available to one skilled in the art may be used or carried out in place of or in combination with those disclosed herein, and are considered to fall within the scope of the present disclosure.

As used in the present disclosure, the term “treating” or “treatment” refers to an intervention performed with the intention of preventing the development or altering the pathology of a disease or infection. Accordingly, “treatment” refers to both therapeutic treatment and prophylactic or preventative measures. A therapeutic agent may directly decrease the pathology of a disease or infection, or render the disease or infection more susceptible to treatment by other therapeutic agents or, for example, the host's immune system. Treatment of patients suffering from clinical, biochemical, radiological or subjective symptoms of a disease or infection may include alleviating some or all of such symptoms or reducing the predisposition to the disease. Improvement after treatment may be manifested as a decrease or elimination of such symptoms. Thus, the compositions are useful in treating a condition by preventing the development of observable clinical symptoms from infection, and/or reducing the incidence or severity of clinical symptoms and/or effects of the infection, and/or reducing the duration of the infection/symptoms/effects.

“Infections” as used herein refers to any microbial invasion of a living tissue that is deleterious to the organism (host). Microbial infections may be caused by microorganisms, or “infectious agents,” including, but not limited to, a bacteria, virus, fungus, parasite, protozoan, helminth, or prion. Similarly, the term “disease” refers to any pathological condition and includes the overt presentation of symptoms (i.e., illness) or the manifestation of abnormal clinical indicators (e.g., biochemical indicators). Alternatively, the term “disease” refers to a genetic or environmental risk of or propensity for developing such symptoms or abnormal clinical indicators. An infection or disease is any condition that would benefit from treatment with a molecule according to the present disclosure. This includes chronic and acute disorders or diseases including those pathological conditions which predispose the mammal to the disorder in question.

As used herein, the phrase “effective amount” or “therapeutically effective amount” is meant to refer to a therapeutic or prophylactic amount of conformationally-stable EP67 analog that would be appropriate for an embodiment of the present disclosure, that will elicit the desired therapeutic or prophylactic effect or response, including alleviating some or all of such symptoms of disease or infection or reducing the predisposition to the disease or infection, when administered in accordance with the desired treatment regimen. One of skill in the art recognizes that an amount may be considered therapeutically “effective” even if the condition is not totally eradicated or prevented, but it or its symptoms and/or effects are improved or alleviated partially in the subject. The therapeutically effective dosage of peptide may vary depending on the size and species of the subject, and according to the mode of administration.

References herein to a “conformation” of a peptide or a “conformer” refer generally to the range of geometric orientations/structures/molecular arrangements, and particularly geometric isomers, that a peptide may adopt at a given time. “Conformationally-stable” means that the peptide is generally fixed in a single geometric orientation/conformation/molecular arrangement and not prone to conversion/rotation to a different orientation. In other words, rotation of bonds (particularly between the cis and trans configurations) is restricted or eliminated in the conformationally-stable analogs. Individual residue may also have a “constrained conformation,” which means that they do not undergo cis/trans isomerization.

The term “oligopeptide” refers to a peptide that is at least about 5 amino acids in length and less than 40 amino acids in length. In one embodiment of the present disclosure, the oligopeptide is from about 5 to about 10 residues in length. In one embodiment, the oligopeptide is a decapeptide (i.e., 10 amino acids in length).

As used herein, the term “carboxy-terminal” or “C-terminal” refers to the carboxy-terminus of C5a.

As used herein, the phrase having “selective C5a receptor binding activity” refers to the ability of the analog to bind to CD88 to stimulate the immune-modulatory effect in antigen presenting cells, at the expense of other C5a-mediated inflammatory responses. In other words, binding causes, inter alia, activation of APCs, without directly binding or activating C5a receptor-bearing granulocytes.

As used herein, “concurrent” administration of two therapeutic agents does not require that the agents be administered at the same time or by the same route, as long as there is an overlap in the time period during which the agents are exerting their therapeutic effect. Simultaneous or sequential administration is contemplated (“co-administration”), as is administration on different days or weeks. “Prior” administration refers to administering a conformationally-stable EP67 analog at some time before administering a second therapeutic active agent, irrespective of whether the two therapeutic agents are exerting a therapeutic effect together. Moreover, “following” administration refers to administering a conformationally-stable EP67 analog at some time after administering a second therapeutic agent, irrespective of whether the two therapeutic agents are exerting a therapeutic effect together.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Isomerization of EP67

The synthetic peptide, designated as EP67 (YSFKDMP(MeL)aR (SEQ ID NO:2)), was previously derived from the C-terminal region of human complement component, C5a. An essential amino acid in EP67 is the central proline residue. EP67 has been shown to be effective as a selective C5a agonist, binding specifically to C5aR-bearing macrophages (and other APCs) but not C5aR-bearing neutrophils. EP67 is used in providing activation signals to C5a receptor-bearing macrophages (and other APCs) to mount a robust innate immune response to infection.

EP67 was generated with residue substitutions designed to restrict the conformational flexibility inherent in naturally-occurring C5a₆₅₋₇₄ with the goal of biasing topographical features that might distinguish between C5a-like immune stimulatory responses versus C5a-like inflammatory responses when ligated to the C5aR. In contrast to the highly flexible C5a₆₅₋₇₄, the residue substitutions made in EP67 “lock in” a unique conformational profile that is well accommodated by C5aRs expressed on APCs, but not by C5aRs expressed on inflammatory granulocytes. When EP67 engages C5aR-bearing APCs it induces the release of T helper type 1 (Th1) cytokines (IL-1β, IL-6, IL-8, IL-12, TNFα, IFNγ) from human and mouse macrophages, but few T helper type 2 (Th2) cytokines (IL-2, IL-4, IL-5, IL-10). Also, EP67 induces the differentiation of human and porcine monocytes to macrophages and dendritic cells and decreases necrosis and apoptosis of the macrophages/dendritic cells once generated. Together, these outcomes from the EP67-mediated engagement of C5aR-bearing APCs establish a robust innate immune environment that is capable of reducing/eliminating localized and systemic bacterial, viral, and fungal infections and does so with little or no inflammatory side-effects from engagement of C5 aR-bearing granulocytes.

However, it has been determined that EP67 exhibits a conformational instability at the seventh and eighth positions occupied by Pro and MeLeu.

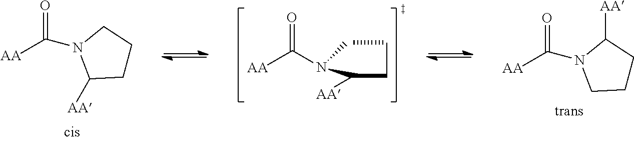

where AA and AA′ are adjacent amino acids in the peptide. This instability undercuts the therapeutic potential of EP67 and its ability to engage C5aR-bearing APCs to create an innate immune environment against resistant (and normal) bacterial infections as well as resistant/normal viral and fungal infections. Overcoming this instability would permit realization of the fullness of the therapeutic potential of the response selective peptides.

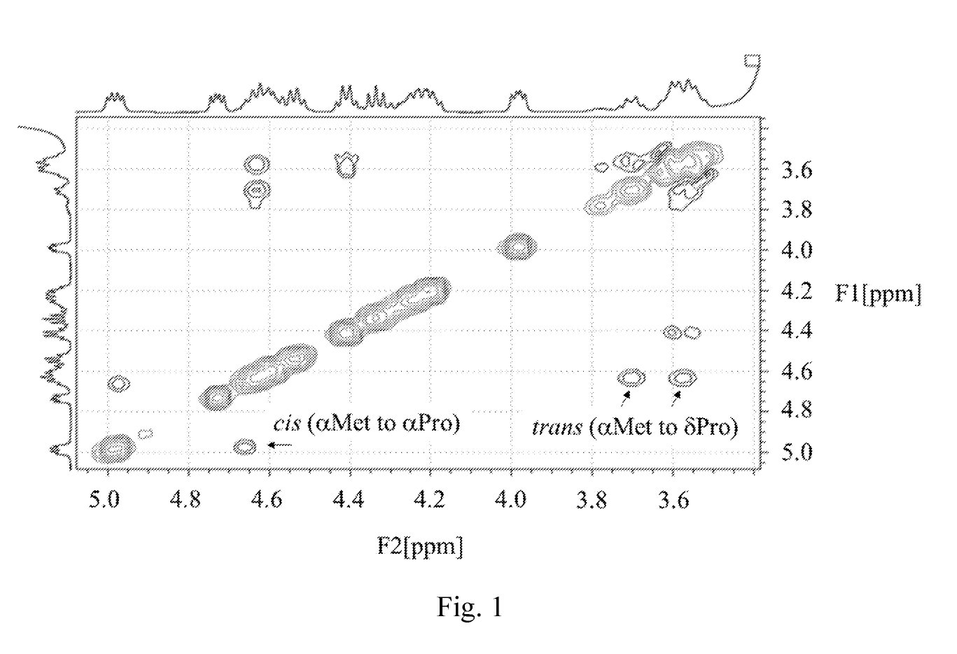

NMR spectroscopy is the best method for detection of cis/trans prolyl isomerization in a peptide. AAₓ-Pro cis/trans isomerization (where AAₓ is any adjacent amino acid residue) is detectable via two resonance frequencies in the NMR spectra per nuclear spin in the proximity of the isomerizing bond. Rotating-Frame NOE Spectroscopy (ROESY) was used to analyze EP67 (150 msec mixing time) in DMSO-d6. Characteristic NOE patterns readily discriminate the resonances arising from the cis and trans conformers allowing for specific assignments of each. Also, the relative populations of each conformer can be determined by integration of the separate peak volumes. Short ¹H-¹H distances (NOEs) between the alpha proton of AAₓ and the alpha proton of Pro are diagnostic of the cis AAₓ-Pro peptide bond. On the other hand, an observed NOE between the alpha proton of AAₓ and the delta protons of Pro are diagnostic of the trans AAₓ-Pro peptide bond.

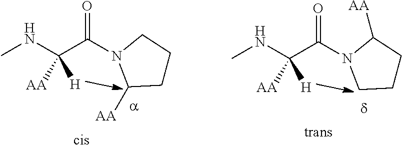

where AA and AA′ are adjacent amino acids. Using these NMR methods, we show that the Met-Pro bond in EP67 (SEQ ID NO:2) exists in both the cis and trans conformations (FIG. 1) resulting in two populations of EP67 conformers in solution in ratios of ca. ˜20% cis and ˜80% trans.

The aim of subsequent work has been on generating analogs of EP67 with a substitution or modification at position 7. More specifically, analogs have been synthesized in which the Pro and Pro-associated cis/trans isomerization is eliminated, while assuring that the synthetic modification or residue substitution retains the biologically important conformational features of EP67 for which the Pro residue was originally introduced. NMR studies on EP67 showed that extended, polyproline II (P_{II})-like backbone conformation in the peptide bond between the Met and Gln residues in C5a₆₅₋₇₄ (ISHKDMQLGR, SEQ ID NO:5) was important biologically. In EP67, a Pro residue was substituted for Gln (Q) because the steric bulk of the cyclic Pro forces extended backbone conformation of the residue immediately to its N-terminus:

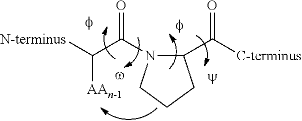

Thus, additional analogs of EP67 will be generated with residues substituted for Pro that exert a Pro-like extended conformation on the peptide bond immediately to their N-termini, but do not undergo cis/trans isomerization.

### Example 2

### Conformationally Stable Analogs of EP67

Conformationally stable analogs of EP67 were synthesized by standard solid phase Fmoc orthoganol methods on appropriately substituted Wang resins. Syntheses were performed on a 0.25-mmol scale and employed the 9-fluorenylmethyloxycarbonyl method of repetitive residue linkages. Peptides were purified by analytical and preparative reverse-phase HPLC on C18-bonded silica columns with 0.1% TFA as the running buffer and 60% acetonitrile in 0.1% TFA as the eluant. Peptides will be characterized by electrospray and MALDI (matrix-assisted laser desorption ionization) mass spectrometry for verification of molecular mass. The analogs are listed in Table 1 below. We have generated two EP67 analogs in which the Pro was substituted with 5,5′-dimethylproline (dmP) (designated as EP145) or 2-aminoisobutyric acid (Aib) (designated as EP144). In EP144, the MeL residue of EP67 was also substituted with Leu. Conformationally stable analogs of EP67

| Designation | Sequence | SEQ ID NO: |
|---|---|---|
| EP144 | YSFKDM(Aib)LaR | 3 |
| EP145 | YSFKDM(dmP)(MeL)aR | 4 |

An inactive control peptide consisting of a scrambled sequence for EP67 was also synthesized for comparison (designated as sEP67, M(MeL)RFPDaYKS (SEQ ID NO:6)).

Analog peptides can be analyzed by NMR spectroscopy to verify structure and ascertain cis/trans conformer ratios of the Pro-substituted analogs. Special attention should be paid to the angles formed about α-carbon (φ and Ψ bonds) and bonds) and carbonyl carbon (Ψ and ω bonds) in the Met residue to the N-terminus of the substituted Pro residues.

The two analogs were generated with the objective of shifting (EP145) or eliminating (EP144) the cis/trans equilibrium of Pro (EP67), yet retaining the Pro-like extension of backbone conformation on the Met residue to its N-terminus. Indeed, substitution of Pro in EP67 with dmP (EP145), altered the cis:trans conformer ratio from 20:80 to 50:50. In contrast and as predicted, substitution of the Pro in EP67 with Aib (EP144) resulted in an all-trans conformation.

### Example 3

### Bioactivity of Conformationally Stable Analogs

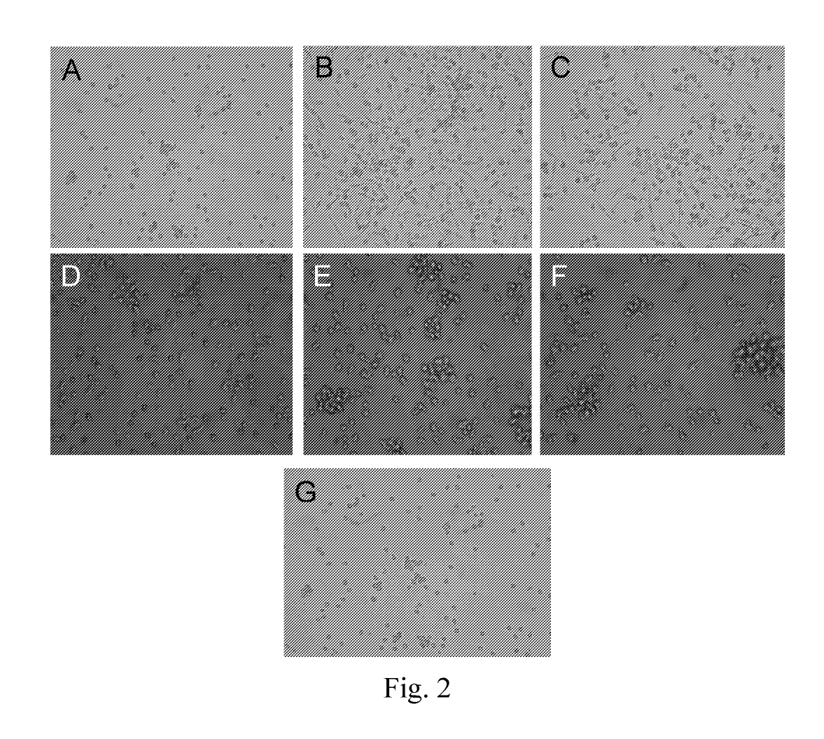

To assess the binding of EP67 analogs to APCs and subsequent biological activity on these cells, peptides were assayed for their ability to elicit the activation of monocytes in vitro and to drive the differentiation/maturation of human monocytes into macrophages and subsequently dendritic cells. As shown in FIG. 2, both EP144 and EP145 drive the differentiation of human monocytes to macrophages in a manner similar to EP67, thus justifying our rationale and approach for further Pro substitutions within this triad region.

Briefly, monocytes were harvested from human peripheral blood mononuclear cells. The human monocytes were incubated at 37 degrees C. for 24 hrs in media the presence of PBS, 100 μg/ml of EP67, 100 μg/ml of EP144, and 100 μg/ml of EP145. The cells were also incubated with for 48 hrs with sEP67 as a control. The cells were then photographed under light microscopy at 20× or 40× magnification. FIG. 2 shows representative bright fields from cells grown in the presence of PBS (Panels A and D), EP67 (Panels B and E), EP144 (Panel C), EP145 (Panel F), and sEP67 (Panel G). Panels A-C & G are at 20× magnification and Panels D-E are at 40× magnification. In media supplemented with EP67, EP144, or EP145, monocytes mature into macrophages exhibiting characteristic phenotype of adhesion to substrate and flattening. Cells grown with the scrambled peptide or PBS do not exhibit this phenotype, but retain the round shape and lack of firm adherence to substrate characteristic of monocytes.

### Example 4

### Additional Conformationally Stable Analogs

Building on the similarities in monocyte differentiation between EP67, EP144 (Aib), and EP145 (dmP), we have designed a small series of structurally diverse EP67 analogs (designated as peptides 1-11) each of which is anticipated to possess a unique conformational ensemble and overall topography. The peptides will have the same basic sequence:
Tyr-Ser-Phe-Lys-Asp-Met-Xaa-(Xaa2)-(D-Ala)-Arg (SEQ ID NO:1)
   Analogs 1 and 2 feature ring expansion and contraction by replacement of Pro with pipecolic acid (#1) or 2-azetidine-carboxylic acid (#2), respectively.

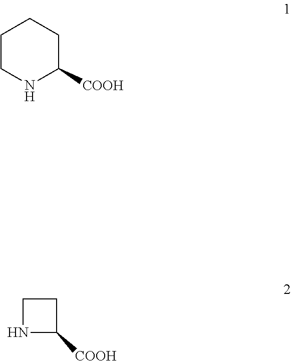 [SMILES: O=C(O)[C@@H]1CCCCN1 ; O=C(O)[C@@H]1CCN1]

Analogs 3-7 feature a variety of conformationally-restricted proline derivatives: 2,4-methano-proline (#3), 2,4-methano-β-proline (#4), 7-azabicyclo[2.2.1]heptane-1-carboxylic acid (#5), 2-azabicyclo[3.1.1]-heptane-1-carboxylic (2,4-methano-pipecolic) (#6), and cis-octahydro-1H-indole-2-carboxylic acid (#7).

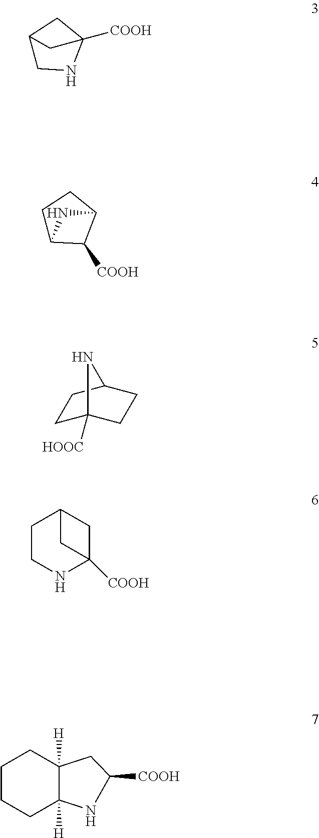 [SMILES: O=C(O)C12CC(CN1)C2 ; O=C(O)[C@@H]1[C@@H]2CC[C@H]1N2 ; O=C(O)C12CCC(CC1)N2 ; O=C(O)C12CC(CCN1)C2 ; O=C(O)[C@@H]1C[C@@H]2CCCC[C@@H]2N1]

Notably, amino acid building blocks for 3, 5, and 6 are achiral.

Analog 8 features an N-methylalanine ring-opened substitute of Pro, whereas analog 9, with its 1-aminocyclohexane-carboxylic acid, is a spiro version of the 2-aminoisobutyric acid-containing analog (EP144).

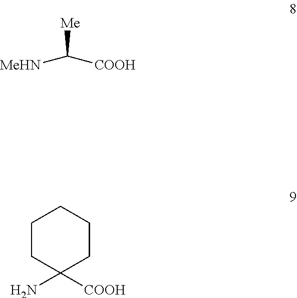 [SMILES: CN[C@@H](C)C(=O)O ; NC1(C(=O)O)CCCCC1]

Finally, the 1,5-tetrazole (#10) and N-tert-butyl glycine (#11) analogs will both exist in fixed cis conformations that will closely approximate the EP67 Pro cis conformer.

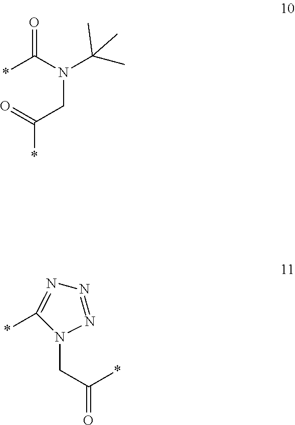 [SMILES: *C(=O)CN(C(*)=O)C(C)(C)C ; *C(=O)Cn1nnnc1*]

Based on the data obtained for peptide analogs 1-11, alternate Pro analogs can be considered including 2-azabicyclo-[2.2.1]heptane-3-carboxylic acid, 7-azabicyclo[2.2.1]-heptane-2-carboxylic acid (2,5-ethano-β-proline), the achiral 2-azabicyclo [2.2.2]octane-1-carboxylic (2,5-ethanopipecolic acid) and 9-azabicyclo-[3.3.1]nonane-1-carboxylic (2,6-propanopipecolic acid) (21), 3,4-phenylproline, 2-amino-adamantane-2-carboxylic acid, and N-methylleucine.

The peptides will be synthesized by standard solid phase methods using Fmoc orthogonal methods on C-terminal (Arg) substituted Wang resins. N-(fluorenyl-9-methoxycarbonyl) (Fmoc) protected amino acid precursors for 1, 2, and 6-9 are commercially available and can be used directly in place of Fmoc-Pro for the synthesis of the corresponding target decapeptides. Fmoc derivatives of amino acid precursors for 3, 4, and 5 can be obtained by reaction of the corresponding amino acids with either 9-fluorenylmethyl dimethoxytriazinyl carbonate (Fmoc-DMT) or 9-fluorenylmethyl benzotriazole. The amino acid building blocks for 3, 4, and 5 can be obtained by procedures described in the indicated references. The synthesis of target decapeptide 10 will require synthesis of the Fmoc tripeptide precursor C:

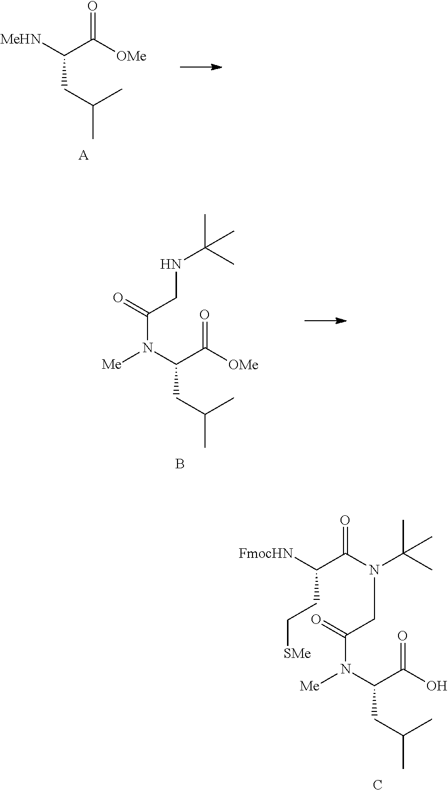

Acylation of N-MeLeu methyl ester A with bromoacetylbromide followed by N-alkylation with tert-butyl amine will yield. Acylation of B with the acid chloride (or acid bromide) of FmocMet followed by hydrolysis of the methyl ester will yield the needed precursor C.

Target decapeptide 11 will require synthesis of the Fmoc tetrazole precursor E, which can be obtained by successive treatment of Fmoc dipeptide methyl ester D with triphenylphosphine, diethyl azodicarboxylate, and azidotrimethylsilane followed by aqueous potassium carbonate:

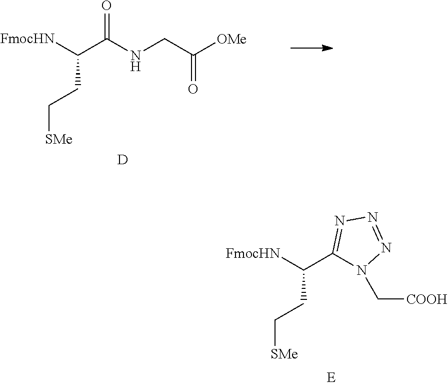

Peptides will be purified by standard HPLC methods using C₁₈-bonded silica analytical and preparative columns. Peptides will be characterized by electrospray and MALDI (matrix-assisted laser desorption ionization) mass spectrometry for verification of molecular mass. Also, analogs will be will be analyzed by NMR spectroscopy to verify structure and ascertain cis/trans conformer ratios of the Pro-substituted analogs. Special attention will be paid to the angles formed about α-carbon (φ and ψ bonds) and carbonyl carbon (ψ and ω bonds) in the Met residue to the N-terminus of the substituted Pro residues.

The eleven, full-length (decapeptide) EP67 analogs generated with the Pro substitutions in FIG. 4 will be assessed for: 1) their binding affinity to C5aRs on human and porcine macrophages and neutrophils, 2) their potency in these cells as measured by cytokine release from macrophages and myeloperoxidase (MPO) release from neutrophils, and 3) from these two binding affinity and potency assays, their bioselectivity relative to EP67 and natural C5a.

1. C5aR Binding Affinity. Analog binding affinity to the C5aR will be determined on C5aR-bearing macrophages and neutrophils by binding site competition with ¹²⁵I-labeled C5a in accordance to our previously published methods (Taylor et al. Development of response selective agonists of human C5a anaphylatoxin: conformational, biological, and therapeutic considerations. Curr. Med. Chem. 8:675-684, 2001; Vogen et al. Differential activities of decapeptide agonists of human C5a: the conformational effects of backbone N-methylation. Int. Immunopharmacol. 1:2151-2162, 2001). C5aR binding affinity will be assessed for each analog by half maximal inhibitor concentration (IC₅₀); i.e., the concentration of analog to inhibit 50% of ¹²⁵I-C5a binding to C5aR. The IC₅₀ of EP67 will be used as a comparative control.

2. Potency. Analog potency will be measured by half maximal effective concentration (EC50); i.e., the concentration of the analog to induce a response halfway between baseline and maximum effect. EC50 values for each analog be determined in two separate assays: cytokine release from macrophages and MPO release from neutrophils.

In macrophages, we will measure the release of the TH1 cytokines IL-6, TNFα, and INFγ in accordance to our previously published methods (Morgan et al. Enhancement of In Vivo and In Vitro Immune Functions by a Conformationally Biased, Response-Selective Agonist of Human C5a: Implications for a Novel Adjuvant in Vaccine Design. Vaccine 28:463-469, 2010; Morgan et al. A novel adjuvant for vaccine development in the aged. Vaccine 28:8275-8279, 2010). These cytokines are consistently released in readily measurable amounts from macrophages (as well as monocytes and dendritic cells) and will be representative of what we have seen with EP67 and consistent with the nature of the supporting data presented above. Briefly, macrophages will be incubated in the presence of various concentrations of analogs (10, 50, 100, 200 μg/ml) in standard cell culture conditions for 6, 12, and 24, hrs. Supernatants will be collected and assayed for the presence and amounts of the TH1 cytokines above using standard ELISA methods. Separate wells of macrophages will be incubated under the same/analogous conditions with EP67 and natural C5a as comparative controls. Macrophages in culture media only (no treatment) will serve as a negative control.

In neutrophils, we will measure the release of the proteolytic enzyme MPO. Briefly, neutrophils will be incubated in the presence of various concentrations of analogs (10, 50, 100, 200 μg/ml) in standard cell culture conditions for 6, 12, and 24, hrs. Supernatants will be collected and assayed for the presence and amounts of MPO using standard ELISA methods. Separate wells of neutrophils will be incubated under the same/analogous conditions with EP67 and natural C5a as comparative controls. Neutrophils in culture media only (no treatment) will serve as a negative control.

3. Bioselectivity. Analog selectivity for cytokine release from macrophages vs. MPO release from neutrophils will be determined by the following equation:

selectivity=antilog[(−Δmacrophage)−(−Δneutrophil)], where:

Δ is the log potency ratio (pD₂ C5a−pD₂ analog) and pD₂ =−log EC₅₀. The “selectivity” of natural C5a will be set at value of 1 using equation since it is equipotent in both cytokine release from macrophages and MPO release from neutrophils. Thus, differences in the potencies between these two C5aR-bearing cells can be assessed relative to C5a; i.e., the greater the value from the above equation, the greater the selectivity relative to C5a. This is the pharmacologically accepted means of determining selectivity between two compounds and was used by us to determine the selectivity of EP67 for engagement and activation of C5aR-bearing APCs over that of C5aR-bearing neutrophils. For example, EP67 has a selectivity factor of 2951 for macrophages activity over that of neutrophils compared to a selectivity factor of 1 for C5a, which is equipotent in both cells.

1. (canceled)

2. A conformationally-stable peptide analog of EP67, having the formula:
(SEQ ID NO: 1)
Tyr-Ser-Phe-Lys-Asp-Met-Xaa-(Xaa2)-(D-Ala)-Arg,
wherein:
Xaa is selected from the group consisting of:
a) alanine;
b) N-methylalanine;
b) 2-aminoisobutyric acid;
c) 3-aminoisobutyric acid;
d) N-methylisoleucine;
e) singly-substituted proline analogs at the 2, 3, 4, and/or 5 positions of the pyrrolidine side chain;
f) doubly-substituted proline analogs at the 2, 3, 4, and/or 5 positions of the pyrrolidine side chain;
g) pseudoproline analog: cysteine-derived thiazolidine, serine-derived oxazolidine, or threonine-derived oxazolidine;
h) trifluoromethylated pseudoprolines;
i) proline analog or homolog having a constrained conformation;
j) trifluoromethylated azetidine 2-carboxylic acid;
k) trifluoromethylated homoserine;
l ) oxetanyl-containing peptidomimetic;
m) N-aminoimidazolidin-2-one analog; and
n) nonchiral pipecolic acid analog: and
Xaa2 is leucine or N-methyl leucine, said peptide having selective C5a receptor binding activity.

3. The conformationally-stable peptide analog of claim 2, wherein said singly- or doubly-substituted substituted proline analogs are 5,5′-dimethylproline, 2,4-methano-β-proline, or 2,5-ethano-β-proline.

4. The conformationally-stable peptide analog of claim 2, wherein said serine/threonine/cysteine-derived pseudoproline analogs are selected from the group consisting of: 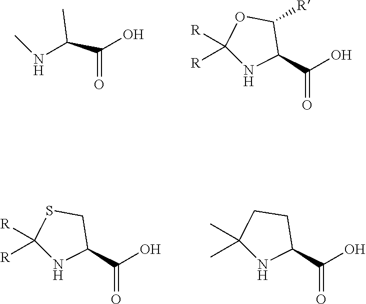
where R and R′═H or CH₃.

5. The conformationally-stable peptide analog of claim 2, wherein said nonchiral pipecolic acid analogs are selected from the group consisting of:

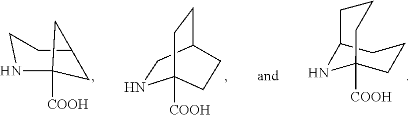 [SMILES: O=C(O)C12CC(CCN1)C2 ; O=C(O)C12CCC(CC1)CN2 ; O=C(O)C12CCCC(CCC1)N2]

6. The conformationally-stable peptide analog of claim 2, said N-aminoimidazolidin-2-one analog being selected from the group consisting of N-amino-imidazolidinone, α-amino-γ-lactam, and an azapeptide.

7. The conformationally-stable peptide analog of claim 2, wherein the EP67 analog is YSFKDM(Aib)LaR (SEQ ID NO:3) or YSFKDM(dmP)(MeL)aR (SEQ ID NO:4).

8. A composition comprising the conformationally-stable peptide analog according to claim 2 dispersed in a pharmaceutically acceptable carrier.

9. The composition of claim 8, further comprising adjuvants, other active agents, preservatives, buffering agents, salts, and mixtures thereof.

10. A method of inducing an immune response against an infection or cancer in a subject, said method comprising administering to said subject a therapeutically-effective amount of a conformationally-stable peptide analog according to claim 2.

11. The method of claim 10, wherein the infection or disease is caused by an infectious agent selected from the group consisting of bacteria, virus, fungus, parasite, protozoan, and prion.

12. (canceled)

13. The method of claim 10, further comprising providing a unit dosage form of said compound dispersed in a pharmaceutically-acceptable carrier prior to said administering.

14. The method of claim 10, wherein said peptide is administered intramuscularly, subcutaneously, intradermally, intranasally, intravenously, orally, or via a transdermal patch.

15. The method of claim 10, further comprising administering an active agent to said subject, said active agent being different from said peptide.

16. The method of claim 15, wherein said peptide and active agent are co-administered.

17. The method of claim 15, wherein said active agent is selected from the group consisting of killed virus, modified live virus, viral or bacterial proteins, viral or bacterial DNA, toxoids, protein subunits, and tumor antigens.

18.-19. (canceled)

20. A kit comprising:
a conformationally-stable peptide analog according to claim 2; and
instructions for administering said peptide to a subject in need thereof.

21. The kit of claim 20, wherein said conformationally-stable peptide analog is provided in unit dosage form.

22. The kit of claim 20, wherein said peptide is provided in a first container, said kit further comprising a carrier in a second container; and instructions for preparing said peptide for administration to said subject.

23. A compound for enhancing an immune response to an immunogenic agent, said compound comprising a conformationally-stable peptide analog according to claim 2 covalently linked to an immunogenic agent.

24. The compound of claim 23, said immunogenic agent being selected from the group consisting of killed virus, modified live virus, viral or bacterial proteins, viral or bacterial DNA, toxoids, protein subunits, and tumor antigens.

25. (canceled)


SEQUENCE LISTING (6 sequences)

SEQ ID NO: 1 - protein, 10 aa, Artificial Sequence
  Synthetic peptide
  MOD_RES (7)..(7): Xaa is Ala, Leu, IsoLeu, Aib, 3ib, dmP, mbP, ebP, MeA, MeL, MeI, substituted Pro analog, pseudoproline, Ser- or Thr-derived oxazolidine, Cys-derived thiazolidine, Tfm azetidine, Tfm homoserine, oxetanyl, Aid, Nai, Agl, Aza, or pipecolic acid
  MOD_RES (8)..(8): Xaa is Leu or N-methyl Leu
  MOD_RES (9)..(9): D-Ala
YSFKDMXXAR

SEQ ID NO: 2 - protein, 10 aa, Artificial Sequence
  Synthetic peptide
  MOD_RES (8)..(8): N-methyl Leu
  MOD_RES (9)..(9): D-Ala
YSFKDMPLAR

SEQ ID NO: 3 - protein, 10 aa, Artificial Sequence
  Synthetic Peptide
  MOD_RES (7)..(7): Xaa is 2-aminoisobutyric acid
  MOD_RES (9)..(9): D-Ala
YSFKDMXLAR

SEQ ID NO: 4 - protein, 10 aa, Artificial Sequence
  Synthetic peptide
  MOD_RES (7)..(7): Xaa is 5,5′-dimethylproline
  MOD_RES (8)..(8): N-methyl Leu
  MOD_RES (9)..(9): D-Ala
YSFKDMXLAR

SEQ ID NO: 5 - protein, 10 aa, Homo sapiens
ISHKDMQLGR

SEQ ID NO: 6 - protein, 10 aa, Artificial Sequence
  Synthetic peptide
  MOD_RES (2)..(2): N-methyl Leu
  MOD_RES (7)..(7): D-Ala
MLRFPDAYKS